(19) United States
(12) Patent Application Publication
(10) Pub. No.: US 2002/0184791 A1
(43) Pub. Date: Dec. 12, 2002

(54) Bathing slipper

(76) Inventor: Po-Chung Ko, Chenping Li (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/878,378

(22) Filed: Jun. 12, 2001

(51) Int. Cl. A61F 5/14; A43B 3/12; A43B 5/08

(52) U.S. Cl. 36/8.1; 36/11.5; 36/141

(57) ABSTRACT

A bathing slipper includes a sole having open spaces, and a vamp having crown-like massaging devices, the massaging devices each formed of a plurality of flexible projecting strips arranged in a circle, the projecting strips each having a conical body and a pointed top tip.

### BACKGROUND OF THE INVENTION

The present invention relates to a bathing slipper, and more particularly, to such a bathing slipper, which is comfortable and safe in use.

FIGS. 1 and 1A show a bathing slipper according to the prior art. This structure of bathing slipper 10 is comprised of a solid sole 11, a vamp 12 fastened to the sole 11, and a brush adhered or stitched to the vamp 12. The brush comprises a flexible sheet material 13 adhered or stitched to the vamp 12, a plurality of cylindrical bristle holders 14 formed integral with the flexible sheet material 13, and bundles of bristles 15 respectively fastened to the bristle holders 14. This structure of bathing slipper has numerous drawbacks as outlined hereinafter.

1. Because the bristles 15 are respectively installed in the bristle holders 14 with labor, the manufacturing process of the bathing slipper is complicated, resulting in a high manufacturing cost.

2. The bristles 15 tend to be forced out of the bristle holders 14 when rubbed against the sole of the user's foot to clean or massage the sole of the user's foot.

3. When rubbing the bristles 15 against the sole of the user's foot, the sole may feel pain.

4. Because the sole 11 is a solid member without open spaces, water tends to be accumulated in the sole.

FIGS. 2A and 2B show another structure of bathing slipper according to the prior art. This structure of bathing slipper 20 comprises a solid sole 21, a vamp 22 fixedly fastened to the sole 21, and an attachment fastened to the vamp 22. The attachment comprises a plastic sheet material 23 and plurality upright plastic pins 24 integral with the plastic sheet material 23. This structure of bathing slipper still has drawbacks. Because the attachment is not extended over the outer surface of the vamp 22, the installation of the attachment destroy the sense of beauty of the bathing slipper, and the limited area of the attachment achieves less massaging effect. Further, because the sole does not have open spaces in it, water tends to be accumulated in the sole.

### SUMMARY OF THE INVENTION

The present invention has been accomplished to provide a bathing slipper, which eliminates the aforesaid drawbacks. According to one aspect of the present invention, the bathing slipper comprises a sole and a vamp integrally injection-molded from plastics. The vamp has crown-like massaging devices formed integral with the outer surface thereof. Each massaging device comprises a plurality of flexible projecting strips arranged in a circle. The projecting strips each have a conical body and a pointed top tip. Because the vamp, the sole and the massaging devices are integrally injection-molded from flexible plastics, the manufacturing cost of the bathing slipper is low. According to another aspect of the present invention, the sole has open spaces in it for quick draining of water, and hollow cylindrical bottom studs for positioning on the floor positively.

### BRIEF DESCRIPTION OF THE DRAWINGS

FIG. 1 is an elevational view of a bathing slipper according to the prior art.

FIG. 1A is an enlarged view of a part of FIG. 1.

FIG. 2A is an exploded view of another structure of bathing slipper according to the prior art.

FIG. 2B is an assembly view of the bathing slipper of FIG. 2A.

FIG. 3 is an elevational view of a bathing slipper according to the present invention.

FIG. 3A is an enlarged view of a part of FIG. 3.

FIG. 4 is an oblique bottom elevation of the bathing slipper according to the present invention.

FIG. 5 is a side view in section of the bathing slipper according to the present invention.

FIG. 6 shows an application example of the present invention.

FIG. 7 shows another application example of the present invention.

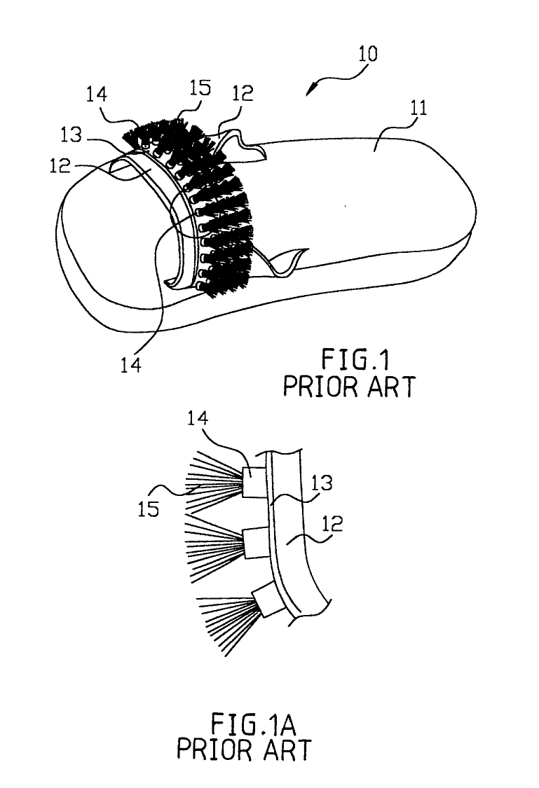

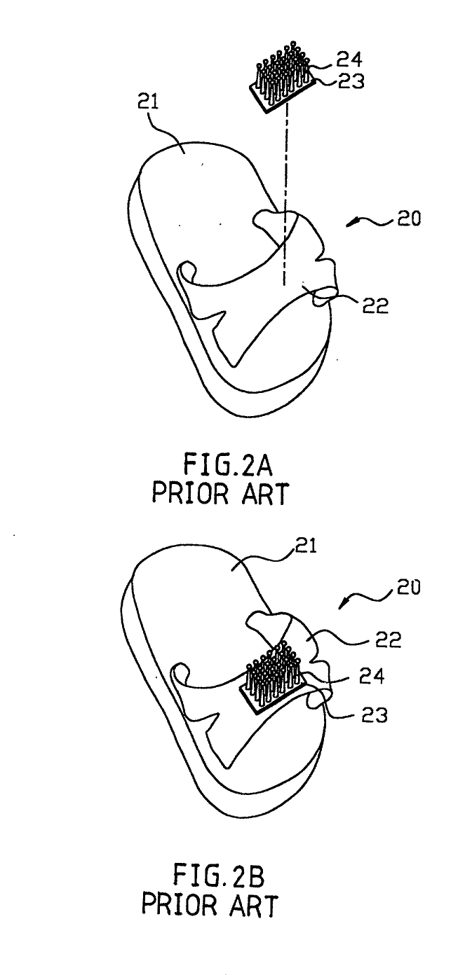

### DETAILED DESCRIPTION OF THE PREFERRED EMBODIMENT

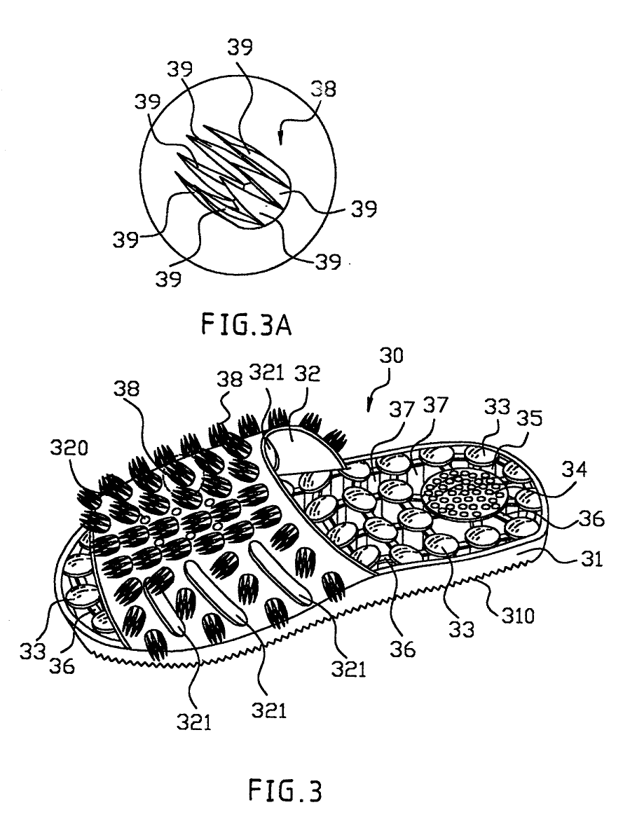

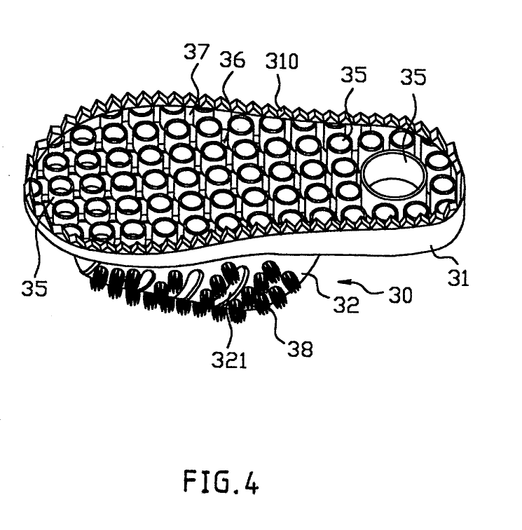

Referring to FIGS. from 3 through 5, a bathing slipper is shown comprised of a sole 31 and a vamp 32 integrally injection-molded from flexible plastics. The vamp 32 has slots 320;321. The sole 31 comprises a plurality of blocks 33;34, a plurality of connecting ribs 36 respectively connected between each two blocks 33;34, a plurality of open spaces 37 respectively surrounded by the blocks 33;34 and the connecting ribs 36, a plurality of cylindrical studs 35 respectively formed integral with the blocks 33;34 at the bottom side and adapted to support the bathing slipper on the floor, and a serrated portion 310 extending around the border of the bottom side thereof. The blocks 33;34 preferably have a convex top surface. The cylindrical studs 35 are hollow and flexible, and adapted to produce friction resistance when touching the floor, preventing the slipper from slipping on the floor. The vamp 32 further comprises a plurality of massaging devices 38. The massaging devices 38 are formed integral with the outer surface of the vamp 32 during injection-molding of the bathing slipper. The massaging devices 38 are shaped like a crown, each comprised of a plurality of flexible projecting strips 39 arranged in a circle (see FIG. 3A). The projecting strips 39 each have a conical body and a pointed top tip.

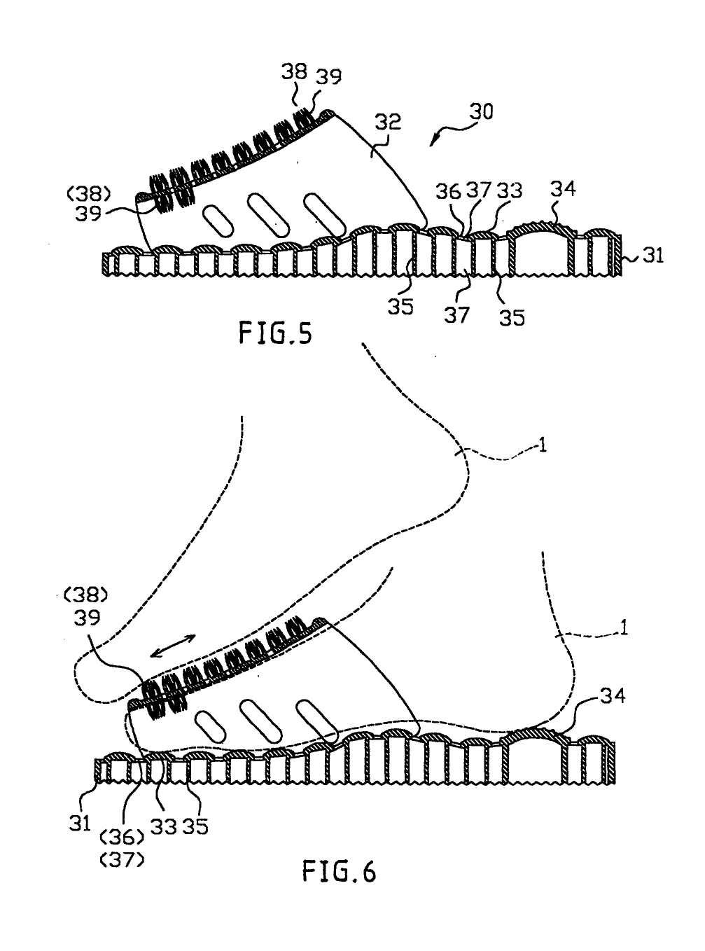

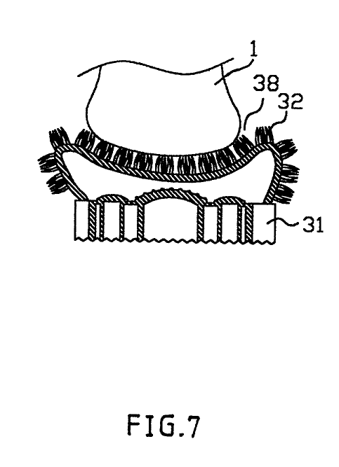

Referring to FIG. 6, the sole 31 of the bathing slipper achieves the following advantages and functions.

1. When the sole of the user's foot is pressed on the blocks 33;34, the convex top surface of each of the blocks 33;34 imparts a massaging effect to the sole of the user's foot.

2. Because the cylindrical studs 35 are hollow and flexible, the bathing slipper is comfortable in use.

3. The blocks 33;34 and the connecting ribs 36 are orthopedically engineered to fit the sole of the foot.

4. The open spaces 37 guide water out of the sole 31, preventing accumulation of water in the bathing slipper.

5. Because the hollow, flexible cylindrical studs 35 are pressed on the floor when the user stands up or walks, much friction resistance is produced between the bathing slipper and the floor, preventing the bathing slipper from slipping on the floor.

6. The serrated portion 310 of the sole greatly increases the friction resistance of the bathing slipper against the floor when walking. of the foot against the flexible projecting strips 39 of the massaging devices 38 to clean and massage the sole of the foot 1 and to stimulate the circulation of flood in the foot 1. Further, the slots 320;321 can have any of a variety of shapes. When rubbing the sole of the foot against the flexible projecting strips 39 of the massaging devices 38, the slots 320;321 drain the water or soap water from the massaging devices 38 and the outer surface of the vamp 32. The massaging devices 38 can also be injection-molded on the inner surface of the vamp 32 along its front side. Because the projecting petals 39 of the massaging devices 38 are flexible and arranged in circles, they are deformed in different directions to fit the curvature of the sole of the foot and to scrap and stimulate the sole of the foot when the user rubs the sole of the foot against the massaging devices 38. Therefore, rubbing the sole of the foot against the massaging devices 38 enable the sole of the foot to be well cleaned and comfortably massaged.

A prototype of bathing slipper has been constructed with the features of FIGS. 3˜7. The bathing slipper functions smoothly to provide all of the features discussed earlier.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A bathing slipper comprising a sole and a vamp, wherein said vamp comprises a plurality of flexible massaging devices formed integral with an outer surface thereof and shaped like a crown, said massaging devices each comprised of a plurality of flexible projecting strips arranged in a circle, said projecting strips each having a conical body and a pointed top tip.

2. The bathing slipper of claim 1 wherein said sole comprises a plurality of blocks, a plurality of connecting ribs respectively connected between each two of said blocks, a plurality of open spaces respectively surrounded by said blocks and said connecting ribs, a plurality of hollow cylindrical studs respectively formed integral with said blocks at a bottom side and adapted to support the bathing slipper on the floor, and a serrated portion extending around the border of a bottom side thereof.
